(19) United States
(12) Patent Application Publication
(10) Pub. No.: US 2022/0370244 A1
(43) Pub. Date: Nov. 24, 2022

(54) METHOD FOR DELIVERING THE FLUID FORMULATION AS A SPRAY OR A JET OF DROPLETS TO A TARGET AREA ON AN EYE

(71) Applicant: Novartis AG, Basel (CH)

(72) Inventors: Matt CHANDLER, Cambridge (GB); Graeme DAVIES, Cambridge (GB); Adrian HORRELL, Cambridge (GB); Rachel MYERS, Cambridge (GB); Grant SMETHAM, Cambridge (GB); Gabrielle WHITWORTH-SMITH, Cambridge (GB); Ben WICKS, Cambridge (GB)

(21) Appl. No.: 17/770,972

(22) PCT Filed: Oct. 30, 2020
(86) PCT No.: PCT/IB2020/060235

§ 371 Date: Apr. 21, 2022

Related U.S. Application Data
(60) Provisional application No. 62/930,871, filed on Nov. 5, 2019.

(51) Int. Cl. A61F 9/00; A61B 5/11; A61B 5/00

(52) U.S. Cl. CPC A61F 9/0026; A61B 5/1103; A61B 5/6844

(57) ABSTRACT

A package (1) for a fluid formulation (2) to be delivered as a spray or a jet of droplets to an eye (4), comprising an enclosed container (5) a storage recess (6) containing a fluid formulation (2); and a delivery recess (7), adjacent to the storage recess (6). The storage recess (6) and the delivery recess (7) are separated by a fluid barrier (8). Package (1) further comprises a matrix of holes (9), for generating the spray and/or jet of droplets, the matrix of holes (9) opening into the delivery recess (7). The storage recess (6) is configured to expel, by application of an impulse thereto, a dose of fluid formulation (2). The matrix of holes (9) is configured to steer the spray and/or jet of droplets to the target area (3) on the eye (4). Also provided is a device (20) for delivery of the fluid formulation, and a method for delivering the fluid formulation (2) as a spray or a jet of droplets to an eye (4) of a user.

### TECHNICAL FIELD

The present invention relates a package for a fluid formulation to be delivered as a spray or a jet of droplets to a target area on an eye according to the preamble of independent claim 1, and more particularly to a blister package designed to store topical ocular medications and to selectively deliver them, finely dispersed, to predefined portions of the eye surface to reach an eye target area.

The present invention also relates to a device for delivery of a fluid formulation as a spray or a jet of droplets to a target area of an eye, adapted to lodge a package as above introduced; as well as to a method for delivering the fluid formulation by such a device.

Such package can be conceived for storing and effectively delivering to a user's eye several drug product formulations, intended for respective indications, such as dry eye disease, generalised chronic ocular surface pain and inflammation, presbyopia etc. The present package and the correlated device are especially developed to contribute to the therapeutic action of the drug products delivered, by enhancing their bioavailability.

### BACKGROUND ART

Currently, patients, or users, dispense topical ocular medications to their eyes by traditional eyedroppers. There is widespread patient dissatisfaction associated with repetitive administration difficulties, which often lead to administration failures, under/overdosing, wastage, and sub-optimal therapeutic outcomes. Normally, patients, or users, are required to tilt their head back, while simultaneously holding open their eye and aligning the output of the eyedropper with the ocular surface. The administration procedure requires positioning the dropper close enough to the face to successfully deliver the drop, but far enough away to avoid contamination or discomfort by making accidental contact between the dropper tip and the ocular surface. This process requires coordination skills which can be demanding. As a result, it occurs that patients end up delivering to the eyelid or cheek instead, or achieve only partial dosing, or deliver more than a single dose, or risk damage/infection of the eye.

The above drawbacks are typically just put up with by patients by resorting to quick fixes such as supplementary application of the ocular medication and use of current reliever medications for a short-term comfort.

The issues related to eyedropper manipulation can be especially taxing for older or disabled patients.

Moreover, current droppers deliver significantly more formulation (30-50 μL) than can be accommodated in the conjunctival sac in normal conditions (7-9 μl). This leads to overspill of the drug formulation onto the face, which is inconvenient. Reflex tearing and increased drainage through the nasolacrimal duct can also ensue.

In particular, drainage of the drug formulation through the nasolacrimal duct is not only a wastage, but it is additionally significant due to the extensive local vasculature which leads to systemic uptake of the drug formulations intended for topical ocular administration. It is known for such a systemic uptake to have led to high-risk complications when treating, for example, glaucoma. In addition to this, the delivery mode is untargeted and does not ensure that the therapeutic action will in fact effectively happen at the areas of cornea and/or conjunctiva which are to be treated.

Conventional eye dropper delivery bioavailability at a target site typically amounts to less than 5% of the delivered dose. Accountable for this low success rate are not only formulation losses to the eyelid, overspill to cheeks or tear duct drainage. Concentrations and residence times at site(s) where therapeutic action is meant are affected by tear layer turnover and blink movements. In fact, conventional eyedroppers deliver slow moving droplets which do not have the ability to penetrate the tear film covering the eye surface, as they struggle to overcome the surface tension and viscosity of the tear film. The droplets dispensed by current eye dropper float on, bounce back from, or ineffectively coalesce on eyes' tear layers. An outer lipid film on the tear layer further aggravates these unwanted outcomes. Ultimately, a medical formulation delivered through current technology actually adversely remains relatively distant from the actual eye surface.

Taking into account economic implications, patients delivering more expensive medications for long-term sustained benefits, such as patients affected by chronic conditions, are naturally affected by administration failures or overspill, as medication wastage results in higher expenses to be covered.

Usually, different eye droppers are required to deliver different formulations and a patient might be confronted with a confusing number and design of dispensing bottles.

Therefore, there is a need for a versatile system for scalable delivery of a variety of fluid formulations to an eye of a patient which allows comfortable, consistent and precise delivery; which minimizes losses to a patient's face and/or tear duct and concurrently promotes selective bioavailability of the fluid formulations just to those areas of the eye which are meant to benefit from the therapeutic effect.

### DISCLOSURE OF THE INVENTION

In embodiments of the invention there is provided a package for a fluid formulation to be delivered as a spray or a jet of droplets to a target area on an eye as it is defined by the features of independent claim 1, by a device for delivery of the fluid formulation adapted to lodge such a package as it is defined by the features of independent claim 24, and by a method for delivering a fluid formulation as a spray or a jet of droplets to a target area on an eye of a user by a device as it is defined by independent claim 41. Further embodiments are described herein, for example, in the dependent claims.

In particular, the invention deals with a package for a fluid formulation to be delivered as a spray or a jet of droplets to a target area on an eye, comprising at least an enclosed container. A matrix of holes is provided on the enclosed container, for generating the spray and/or jet of droplets. The enclosed container comprises a storage recess containing the fluid formulation; and a delivery recess, adjacent to the storage recess.

During storage, the storage recess and the delivery recess are separated by a fluid barrier. The terms “storage” as used herein relates to normal storage conditions over the shelf life of the fluid formulation.

The enclosed container is configured such that the matrix of holes opens into the delivery recess. The fluid barrier keeps the storage recess sterile in normal storage conditions.

The storage recess is configured to expel, by application of an impulse thereto, a dose of fluid formulation beyond the fluid barrier to the delivery recess.

The matrix of holes is configured to deliver the spray and/or jet of droplets to the target area on the eye, and additionally may be configured to steer the spray and/or jet of droplets to the target area. Additionally or alternatively, the matrix of holes may be dimensioned to deliver and/or steer the spray and/or jet of droplets to the target area of the eye.

The term “target area” as used herein in connection with a structure of an eye relates in general to areas of anatomical bodies of the eye. The “target area” initially impacted by the spray and/or by the jet according to the present invention will be an outer surface area of the eye, which is typically externally exposed, e.g. at least a portion of the surface of the cornea and/or of the sclera. It will however be understood that, by effect of improved dispersion, tear film penetration and diffusion through eye tissue as achieved by the present invention, the term target area may also come to more generally encompass parts of anatomical bodies of the eye which are normally covered, in part or totally, and/or internal to the eye. Accordingly, a target area, depending on the indication of the fluid formulation delivered, can be at least an area of the cornea and/or of the sclera and/or of the pupil and/or of the lens and/or of the iris, where the therapeutic action is meant to take place.

In various embodiments, the target area is statistically derived based on anatomical eye features of a sample of users. Advantageously, a statistical shape model for the target area can be built from a dataset collecting eye shape information from a statistically relevant number of users.

Following the above statistical approach, in order to optimize eye coverage over a maximum number of distinct individuals, the hole matrix characteristics can be tailored to match the statistically derived target area, guaranteeing delivery success substantially irrespective of individual eye and lid slot shape.

In various embodiments, the target area can be an oblong shaped area, for instance measuring about 18 mm×3.5 mm.

Alternatively, the target area can be substantially ellipsoidal; or substantially circular; or ring shaped; or distributed over only preselected areas of the eye, like right and left portions of the sclera. Thus, the steering action of the hole matrix can be enhanced to reach different parts of the eye which need to be treated. By way of example, a hole matrix adapted to steer the spray and/or jet on a doughnut-shaped target area can allow to focus treatment on an annular region, or all of, the cornea.

The package can be a disposable package which can be discarded after just one administration of fluid formulation therefrom. The enclosed container may therefore be a unit dose container containing an individual dose of fluid formulation which is delivered in a single application.

On the other hand, the package can be designed to be discarded after administration of a multiplicity of distinct, subsequent doses of fluid formulation therefrom, preferably up to complete use of the volume of fluid formulation contained in the storage recess.

In various embodiments, the package takes the form of substantially a blister receptacle and the storage recess is a blister cavity. In such case, the enclosed container can comprise a base element formed to embed the storage recess; and a cover element, attached at least in part to the base element, closing the storage recess. The storage recess can be produced by deformation of the base element, for instance by cold forming. The base element and the cover element further cooperate to create the delivery recess. The delivery recess can be substantially an expansible pocket, which is created by affixing the base element to the cover element in a way that they are close in contact but not attached over the delivery recess area. The expansible pocket can be, for instance, substantially channel shaped. Such channel would expand when a dose of fluid formulation is expelled beyond the fluid barrier to the delivery recess, upon application of the impulse.

In various embodiments, the base element and the cover element are made of aluminium laminate material, the aluminium layers advantageously providing a gas and/or water vapour barrier. The base element can be a thicker layer of aluminium laminate material than the cover element, for instance the base element can be 138 micrometers, suitable for cold forming, whereas the cover element can be 98 micrometers.

In various embodiments, the inner walls of the enclosed container are created by a layer of polyethylene which contacts the fluid formulation. Polyethylene is advantageously stable with a wide array of formulations.

In various embodiments, the enclosed container comprises a permanent seal between the base element and the cover element. The permanent seal can be created along the outer periphery of the storage recess and of the delivery recess.

The storage recess can be made collapsible by application of the impulse. In this case, the impulse is preferably a pressure impulse. The storage recess can consequently be shaped and/or structurally dimensioned to collapse by folding into itself under the pressure impulse, so that the force required to crush the blister cavity is reduced and the residual volume of fluid formulation remaining trapped therein is minimized.

In various embodiments, the fluid barrier is a frangible seal, positioned between the base element and the cover element, which breaks by application of the impulse, such as a pressure impulse. The frangible seal can be interposed between opposite sides of the permanent seal, separating storage recess and delivery recess. Upon rupture of the frangible seal, a dose of fluid formulation is expelled from the storage recess to the delivery recess.

In various embodiments, the permanent seal and/or the frangible seal is a heat seal. The heat seal can be for instance created by heating two adjoining polymer layers within the structure of the aluminium laminate material. The heating temperatures, as well as the time the heating temperatures are applied for, can be adjusted to selectively create the permanent seal and the frangible seal. The frangible seal can thus be created by heating the polymer layers to an extent that only a relatively small amount of diffusion happens therebetween, whereas the permanent seal can result from a deeper entanglement of polymer chains of the polymer layers, ensuing intermolecular diffusion.

In various embodiments, the heat seal is created ultrasonically. This technology advantageously allows for less heat migration to the enclosed container of the package, particularly to the storage recess. The fluid formulation therein contained is thus advantageously spared any heat induced alteration.

On the other hand, the fluid barrier can be instead reversible and usable multiple times for delivery of respective doses. Accordingly, the fluid barrier can be an elastic membrane positioned on an aperture between storage recess and delivery recess. The elastic membrane can thus be configured to deflect and let fluid pass between storage recess and delivery recess upon any application of an impulse, and then to go back to a sealing position, obstructing the aperture. The fluid barrier can also alternatively be a miniaturized one-way valve, such as a miniaturized diaphragm valve.

In various embodiments, the storage recess contains a volume of fluid formulation comprised between 1 and 50 microliters. Even more preferably, particularly in case of highly advanced, effective, or concentrate drug products, the storage recess contains a volume of fluid formulation comprised between 5 and 18 microliters. Thus, wastage can be minimized, while coverage of the targeted eye surface is guaranteed without overloading the conjunctival sac.

If the fluid formulation is oxygen sensitive, the storage recess can further comprise a volume of 5 to 10 microliters of an inert gas, such as nitrogen. Alternatively, the storage recess can be filled under vacuum.

In various embodiments, individual holes of the matrix are configured to deliver respective spray or jet droplets to respective portions of the target area of the eye. This preferential delivery mode can be employed to refine and adjust the coverage of the intended targeted eye surface.

Moreover, the number and/or the diameter of holes of the matrix can be adjusted so that the spray or jet of droplets delivered matches a preset shape of the target area. In fact, the shape of the spray or jet can be designed to match the shape of the required eye target area by adjusting such hole characteristics. Besides, further hole characteristics such as the pattern of the matrix, the shape of the holes and/or the thickness of the material used to manufacture the portion of the enclosed container integrating the holes can be fine-tuned to achieve that the shape of the spray or jet matches the shape of the required eye target area.

In various embodiments, the number and/or the diameter of holes of the matrix is adjusted to obtain corresponding droplet sizes and/or droplet velocities. By adjusting the number and/or the diameter of holes, the flow rate of a given volume of fluid formulation passing through the holes can be controlled, thus achieving a desired duration of the delivery event, particularly such that a blink reflex is not triggered. As a result, it can be ensured that completion of delivery happens before a blink reflex is triggered. Normally, the reflex time between a trigger and a blink is approximately 100 milliseconds. Therefore, the number of holes and their size can be tailored, for a specific fluid formulation, to ensure full delivery of the given volume in a timeframe lower than 100 milliseconds, for instance within 50 milliseconds.

Furthermore, by tailoring droplet sizes and droplet velocities, a proportional penetration in the tear layer is possible, increasing bioavailability of the dispensed fluid formulation. Highly energetic droplets end up being encapsulated deep within the tear layer, thus providing higher concentrations and residence time of the active pharmaceutical ingredient at the intended sites of therapeutic action at the cellular surface of the eye tissues. The effectiveness of the fluid formulation is consequently improved, the active principle reaches the eye tissues rather than being washed away by blink actions.

To further customize the delivery, even individual holes of the matrix may be configured to obtain corresponding droplet sizes and/or droplet velocities for delivery to respective portions of the target area of the eye.

In order to fine-tune the delivery, the number and/or the diameter of holes can be adjusted to physical properties of fluid formulation to be delivered and/or to chemical properties thereof. Physical properties of the fluid formulation comprise viscosity, surface tension and rheological (e.g. shear thinning or thickening) properties. For formulations comprising larger molecules liable to shear, hole diameters will be selected to be proportionally large.

Preferably, the matrix of holes is created by a femtosecond laser. Such a high speed laser enables tight hole pitch, increases throughput and allows for a low cost per package. Femtosecond laser micromachining also vaporises material, which advantageously reduces the risk of particulates and thermal damage to the package material, such as aluminium laminate foils, during drilling. At any rate, the strength of the material employed for the package, e.g. of the aluminium laminate material, influences the hole pitch which is admissible without incurring in adverse doming effects, that is in a domed deformation of the material around the hole during a spray or jet event.

In various embodiments, the package comprises 1 to 500 holes. If, based on patient acceptance preferences, on characteristics of the package material and of the fluid formulation dispensed, a fine mist of very small and relatively slow particles is desirable, a proportionally higher number of holes can be created. Taking into account all instances, on average, the number of holes can be comprised in a range of 8 to 40 holes.

The holes have diameters which can be comprised in a range of 1 to 600 micrometers. A hole diameter on the higher end of the range will be more appropriate for very viscous, dense fluid formulations. Preferably, the hole diameter is comprised in a range of 20 to 300 micrometers. Understandably, for a given volume of fluid formulation to be delivered and a maximum time for full delivery desired, for instance in the order of 50 milliseconds, a smaller diameter for the holes will conversely imply a higher number of holes. In designing the matrix, the number of holes can be adjusted so that good steering to the target area is still achieved.

In various in various embodiments, the holes may occupy an area from about 0.1 to 2 mm², such as 0.5 to 0.9 mm². However, it is to be noted that hole density is not necessarily an important parameter in delivering and/or steering fluid to the eye.

By way of example, tests have shown that, when delivering a small volume of 15-18 microliters of fluid formulation having a viscosity of 1 cP, a matrix of 36 holes having a diameter of 30 μm can be effectively employed to complete delivery by a time of approximately 50 milliseconds, i.e. well before a blink reflex. With a fluid formulation having a viscosity of 10 cP, a matrix of 20 holes having a diameter of 40 μm can be successfully employed to deliver a comparable small volume within a blink reflex. With a fluid formulation having a viscosity of 100 cP, a matrix of 16 holes having a diameter of 60 μm can be effectively employed to deliver a similar volume within a blink reflex.

When considering the possibility of a number of enclosed containers adjusted to represent a dosing regimen, such as a daily dosing regimen, a plurality of enclosed containers can be packed to each other on a common support. For instance, four enclosed containers can be packed together, each for one of two eyes of a patient, for a treatment of two daily doses. This configuration can achieve space optimisation. Conveniently, depending on the dosing regimen cycle and on the overall duration of the treatment, several configurations of a multiplicity of enclosed containers can be created on a common support.

In the case as above introduced, the package can be substantially shaped as a disk, wherein a plurality of enclosed containers is arranged on the disk so that each respective matrix of holes is oriented substantially towards the center of the disk. The disk can be round or oblong, possibly provided with profiles which function as indexing aids for positioning the package in a delivery device, e.g. relative to impulse applying means of the device.

The present invention also relates to a device for delivery of a fluid formulation as a spray or a jet of droplets to a target area of an eye, adapted to lodge a package as above described.

The device comprises impulse applying means for applying an impulse to a storage recess of an enclosed container of the package positioned at a dosage station. At the dosage station, following an impulse, a dose of fluid formulation can be expelled out of the enclosed container to create a spray or a jet of droplets.

The device also comprises position control means for positioning the enclosed container of the package relative to the impulse applying means; as well as registering means for placing the device at an appropriate distance from the eye and/or for aligning the device with the target area on a user's eye. The alignment can also be achieved between an outlet of the device for letting the spray or the jet of droplets out of the device and the target area on the user's eye. Alternatively, either the device or the device outlet can be aligned with one of the eye's anatomy features, such as a pupil. Ultimately, in the latter case, the specific anatomy features, such as the pupil, come to be the target area.

In various embodiments, the device is adapted to lodge a package comprising a plurality of enclosed containers. In such case, the position control means comprise indexing means to feed in succession each of the enclosed containers to the dosage station wherein a dose of fluid formulation is expelled out of the enclosed container to create a spray or a jet of droplets.

The impulse applying means may comprise an electromechanical device and preferably comprises a motor which also drives the indexing means. Thus, two functions can be efficiently correlated and automated, space optimised and the overall dimensions of the device can be contained. This is advantageous for a hand-held device intended for users with some health condition.

Preferably, the impulse applying means is automatically resettable to a reset position after the application of the impulse.

The impulse applying means can be a pressure impulse means comprising a firing member configured to compress the storage recess of an enclosed container. The firing member can be dimensioned relative to the storage recess so as to leave minimum residual volume of fluid formulation therein. The firing member can take several forms and can be, for instance, substantially cylindrical or disc-shaped. Depending on the amount of the impulse to be applied and on a desired profile of the corresponding force, alternative designs for the firing member can be envisaged, such as a sliding wedge or a hinged plate, adapted to respectively slide or hingedly rotate to execute a compression of the storage recess of an enclosed container.

The initial amount of the imparted impulse can be adjusted, for instance, by adjusting the size of the gap between the firing member and the storage recess in the reset position. The gap width is proportional to the impulse initially generated by firing. Thus, a faster initial spray and/or jet of droplets can be generated by increasing the gap between the firing member and the storage recess at the reset position. At least the initial flow rate of fluid formulation can also be enhanced by a proportionally higher amount of pressure applied, taking into due consideration the acceptability of the spray force as perceived by a user.

Preferably, the impulse applying means comprises a cam mechanism and a motor coupled thereto. Relative to such an embodiment, the cam mechanism comprises a substantially circular profiled cam portion and a piston assembly. The piston assembly can be resiliently biased against the profiled cam portion and rotatively coupled to the profiled cam portion by the motor. The overall mechanism can be thus conceived so that in a fired angular position of the piston assembly relative to the profiled cam portion, the firing member of the piston assembly applies a pressure impulse on an enclosed container of the package positioned at the dosage station. In a reset angular position of the piston assembly relative to the profiled cam portion, the firing member is instead retracted and automatically brought to a reset position for a subsequent firing.

Preferably, the piston assembly comprises a driver segment, and a follower segment comprising a follower member and the firing member. In this case, the driver segment and the follower segment are mutually slidably engaged, to move relative to each other along a longitudinal axis of the piston assembly, and resiliently biased by way of a contrast spring packed therebetween. Thus, the follower segment rotatively contacts, by the follower member, the profiled cam portion, to sequentially pass from the reset angular position to the fired angular position of the impulse applying means. Preferably, in the reset angular position the contrast spring is fully retracted; whereas in the fired angular position the contrast spring is fully released.

The contrast spring can exert a force comprised in a range of 1 N to 100 N. Preferably, the spring force is comprised between 22 N and 60 N. By being at least 22 N, the spring force exceeds a minimal storage recess or blister crush force encountered to let fluid formulation disperse by at least a safety threshold. By being limited to 60 N, the spring force advantageously complies with a user's spray acceptability and allows to keep mechanical forces and spring weight manageable on a hand held device.

Preferably, the indexing means comprises a turntable for lodging the package, such that, substantially between angular positions of the piston assembly relative to the profiled cam portion wherein the firing member is retracted up to the reset angular position, the turntable comes to be rotatively coupled to the motor of the impulse applying means and is thereby driven in rotation to bring a next enclosed container to the dosage station.

Preferably, the registering means comprises a telescopic eyecup means configured to rest on anatomical features surrounding the eye of a user. Such telescopic eyecup means can be removable. Alternatively, or even in addition thereto, compliant eyecup means can be provided, configured to conform to anatomical features surrounding the eye of a user. In general, these eyecup means can have a stabilising function.

Preferably, the device comprises a controller for the processing of electronic signals coupled to the impulse applying means, namely to the motor. The provision of a reflectance proximity sensor means, coupled to the controller, allows to determine phases of an eye blink cycle, particularly an eye opening phase. To this purpose, the reflectance proximity sensor mean can comprise an emitter unit and a receiver unit. The emitter unit is configured to send out a beam of light to the user's eye and the receiver unit is configured to detect a corresponding beam of light reflected therefrom and to transmit to the controller an electronic signal based on the reflected beam of light.

More specifically, the controller can be configured to determine a rate of change in the reflected beam of light, which depends on the current reflectance of the eye. Based on the rate of change, the controller can be configured to determine an eye opening phase of an eye blink cycle. Thus, during the eye opening phase, the controller can be configured to produce a delivery activation signal executable on the impulse applying means. To dynamically determine an eye opening phase from start of the opening process is advantageous with respect to simply, more statically determining an eye open state, since the latter determination can be belated enough to hinder a prompt and complete delivery of a fluid formulation dose.

Preferably, the reflectance proximity sensor means is an infrared sensor. Infrared technology is beneficial because it does not trigger a blink reflex, as infrared light not visible. Moreover, an infrared sensor is not ‘confused’ by the amount of ambient light, it works with a wide variety of skin pigmentation levels, and, when used at low power, it is also completely safe.

In various embodiments, the device is configured to determine the eye opening phase of an eye blink cycle, to activate the impulse applying means and to complete delivery of the fluid formulation as a spray or a jet of droplets to the target area of the eye within 100 milliseconds, preferably within 70 milliseconds. The duration of a spontaneous human blink is typically about 100-400 milliseconds. If a reflex blink is triggered by the dose being delivered to the eye, the reflex time between a trigger and an actual blink is approximately 100 ms. Thus, the above configuration ensures the minimum time window available for delivery. Even when intentionally blinking twice, there is still a similar time window available for delivery.

Incidentally, human reaction time to an auditory stimulus is in the region of 250 milliseconds. If a full delivery to the target area of the eye is obtained within 100 milliseconds at the most, the user will not be adversely affected by any sound emitted by the device mechanism during actual application of the treatment.

The device according to the present invention does not require channeling or conducting means, as the package lodged is inherently provided with a hole matrix able to steer the spray and/or jet of droplets to the target area on the eye.

The device preferably simply comprises an outlet for letting the spray or the jet of droplets out of the device, the outlet having a central axis. Accordingly, the registering means can comprise an offset viewing orifice (substantially aligned with the central axis of the outlet; and a backplate comprising visual cues. The offset viewing orifice and the backplate are configured so that, at an appropriate distance and/or in an aligned state, the visual cues are visible through the offset viewing orifice in a predefined pattern and/or a predefined relation thereto.

The offset viewing orifice may be integrated in a front plate which may comprise visual cues for aligning the device horizontally.

In addition to, or in alternative to, the above registering means, the device can be provided with other alignment aids for aligning the device, or an outlet thereof, with the target area of a user's eye, or with a pupil thereof. Such alignment aids can comprise a mirroring surface configured to reflect a device user's eye; and/or a colored source of light and/or a high contrast target such as a LED, preferably at the end of a conducting tube; and/or a through hole, configured to guide a user to look in a target direction; and/or accelerometers, gyroscopes, magnetometers and/or triangulation sensors.

In various embodiments, the device according to the present invention comprises data transmission and/or reception circuitry for communication with a mobile hand-held communication device. The mobile hand-held communication device can then be provided with an application program executable thereon.

Data transmission and/or reception and/or communication circuitry can be wired or preferably wireless. Such communication circuitry can comprise any as known to the art, and may be long or short range, low medium or high energy, RF or optical and others. Examples include a GPS communications circuit, a mobile broadband circuit, a Bluetooth modem and similar.

Algorithms integrated in the application program can be any which are useful to monitor or improve patient compliance, understanding, operations, safety, efficacy, systems analysis and data collection. Dose tracking algorithms and/or adherence algorithms are frequently employed examples. The algorithm functionalities can comprise sending out smart dose reminders e.g. location or activity based; and/or delivered dose confirmation functionalities. Dose refill reminders can also be included in a way that behavioral aspects of adherence are envisaged and addressed. Further algorithm functionalities can also be information modules focused on the fluid formulation to be administered, e.g. posology per eye of the given formulation; or modules allowing the user to determine an own reading prescription, or user's eye and vision characteristics in general. Data exchanged between mobile hand-held communication device and the device according to the present invention can therefore relate to all of the above, including dose counting, refill messages and formulation properties.

The present invention also relates to a method for delivering a fluid formulation as a spray or a jet of droplets to a target area on an eye of a user by a device such as the device above described. The method according to the present invention can also be applied to devices which, though structurally modified from the specific embodiments described in detail in the following, can be similarly actuated to carry out the abovementioned functions.

In particular, while reference has been made to pressure impulse applying means of the device, comprising a firing member configured to compress the storage recess of an enclosed container, the method according to the present invention can also be advantageously applied to devices which comprise differently configured extraction means for extracting the fluid formulation from a container lodged in, or coupled to, the device.

Accordingly, these extraction means can comprise a different typology of impulse applying means for applying an impulse to an enclosed container of fluid formulation lodged in the device. For instance, the device can be provided with a piezoelectric fluid ejection means configured to dispense the content of a fluid formulation filled container. In this case, a piezoelectric transducer converts electrical energy into extremely rapid mechanical vibrations. By way of example, ultrasonic oscillations can be generated and transmitted to a portion of the container, to produce cycles of acoustic pressure in the fluid formulation and consequently determine the ejection of a spray or a jet of droplets thereof.

The man skilled in the art will also understand that the method according to the present invention can be applied for delivering a fluid formulation as a spray or a jet of droplets out of devices provided with yet different extraction means for extracting the fluid formulation from a container lodged in, or coupled to, the device. For instance, by appropriately adapting the actuation of the extraction means, the present method can be implemented in devices which eject a spray or jet of fluid formulation thanks to:
an incorporated Venturi system, wherein a reduction in pressure in a reduced area section of a duct can be used to “suck” the fluid formulation from a reservoir coupled to the device into an air stream; or
a mesh nebulization system of the device, forcing the fluid formulation through a mesh, either static or vibrating, to create a mist of droplets; or
a pressure swirl atomizer nozzle of the device, wherein the fluid formulation is made rotate within a swirl chamber to form a thin, conical sheet which breaks into discrete particles upon exiting to the ambient environment; or
an aerosol dispenser.

Combinations of the various abovementioned extractions means can also be envisaged and still be compatible with the claimed method.

Similarly, the method according to the present invention can be implemented substantially independently from the typology of the container of fluid formulation lodged in, or coupled to, the device. In fact, while in the following an embodiment will be presented wherein a package lodged in the device comprises an enclosed container in the form of a unit dose blister, it will be appreciated that a different kind of container of fluid formulation can be lodged in, or coupled to, the device.

Thus, the container of fluid formulation can also take the form of an ampoule or a reservoir, such as a bulk liquid reservoir, either internal to the device or otherwise coupled to the device, to be in fluid communication therewith.

The method comprises the steps of sending out a beam of light to the user's eye and detecting a corresponding beam of light reflected therefrom. Additionally, the method comprises a step of determining that the device is within an appropriate distance range from the eye and/or aligned with a target area on the user's eye, or with a pupil thereof (see FIG. 2, element 3). Following this, the method comprises a step of determining that an eye blink cycle is occurring, based on a rate of change in the intensity of the reflected beam of light; and a step of determining an eye opening phase of the eye blink cycle.

Once the eye opening phase of the eye blink cycle has been established, the method comprises a step of transmitting a delivery activation signal to the extraction means, during the eye opening phase. A step of delivering the fluid formulation as a spray or a jet of droplets to the target area of the eye within a predefined time from the end of the eye opening phase ensues.

Preferably, determining that the device is within an appropriate distance range from the eye comprises the step of measuring a value of the reflected beam of light.

If the value of the reflected beam of light is lower than a first minimum intensity threshold value, the method can comprise the step of assuming the device is not within an appropriate distance range from the eye and not proceeding to the next step of determining a rate of change in the reflected beam of light.

If the value of the reflected beam of light is instead higher than the first minimum intensity threshold value, the method can comprise the step of determining if the value of the reflected beam of light maintains above the first minimum intensity threshold value for a time longer than a minimum time threshold.

If the value of the reflected beam of light does not maintain above the first minimum intensity threshold value for a time longer than the minimum time threshold (F), the method can comprise a step of not proceeding to the next step of determining a rate of change in the reflected beam of light.

If the value of the reflected beam of light maintains instead above the first minimum intensity threshold value for a time longer than the minimum time threshold, the method comprises a step of determining a rate of change in the reflected beam of light.

Preferably, determining that an eye blink cycle is occurring comprises the step of determining an eye closing phase, followed by a step of determining an eye opening phase.

Preferably, determining the eye closing phase comprises the steps of measuring the current the value of the reflected beam of light; measuring the minimum value of the reflected beam of light within a refresh time equal to the maximum length of time within minimum and maximum values of the reflected beam of light are updated; calculating a difference between the current value and the minimum value of the reflected beam of light as above measured; and establishing that the difference is at least a threshold value. Finally, the determination of the eye closing phase is complete if also it is determined that the rate of positive change is higher than a minimum positive gradient threshold.

Preferably, determining the eye opening phase of the eye blink cycle comprises the steps of measuring the current the value of the reflected beam of light; measuring the maximum value of the reflected beam of light within a refresh time equal to the maximum length of time within which minimum and maximum values of the reflected beam of light are updated; calculating a difference between the current value and the maximum value of the reflected beam of light as above measured and establishing that the difference is at least a threshold value. The determination of the eye opening phase is complete then if also it is determined that the rate of negative change is higher than a minimum negative gradient threshold; in combination with a step of verifying that all the conditions above are met within a preset maximum blink time from the eye closing phase.

### BRIEF DESCRIPTION OF THE DRAWINGS

The package, the device and the method according to the invention are described in more detail herein below by way of exemplary embodiments and with reference to the attached drawings, in which:

FIG. 1 shows a section view of an embodiment of a package according to the present invention;

FIG. 2 shows a schematic view of three exemplary target areas on an eye, to which the spray and/or jet of droplets created by the package of FIG. 1 can be steered;

FIG. 3 shows a lateral view of a first embodiment of a device according to the present invention, adapted to lodge the package of FIG. 1, as it is employed by a user for delivering a spray or a jet of droplets to a target area of his eye;

FIG. 4 shows a frontal view of the device of FIG. 3, displaying an outlet of the device for letting the spray or the jet of droplets out of the device, aligned with a target area on the user's eye;

FIG. 5 shows a lateral view of a storage recess of the package of FIG. 1 which progressively collapses by folding into itself under a pressure impulse imparted by a firing member of the device of FIG. 3;

FIG. 6 shows a schematic manufacturing line for the package of FIG. 1;

FIG. 7 is a schematic representation of internal components of a device according to an embodiment of the present invention;

FIG. 8a is a perspective, partially cut away view of the device of FIG. 3;

FIG. 8b is a lateral perspective view of the device of FIG. 3, including a removable cap for the closing thereof;

FIG. 9 is an illustration of a sequence of steps to prepare the device of FIG. 3 for later use, including the steps of lodging a package comprising multiple doses of fluid formulation on position control means of the device; placing a removable eyecup on top of the position control means; and closing the device with a removable cap;

FIG. 10a shows a lateral view of a second embodiment of a device according to the present invention, adapted to lodge the package of FIG. 1, as it is employed by a user for delivering a spray or a jet of droplets to a target area of his eye;

FIG. 10b is a lateral perspective view of the device of FIG. 10a, including a removable cap for the closing thereof;

FIG. 11 is an illustration of a sequence of steps to prepare the device of FIG. 10a for later use, including opening a hinged eyecup for accessing position control means of the device, lodging a package comprising multiple doses of fluid formulation on the position control means; closing the hinged eyecup and placing a removable cap on top to close the device therewith;

FIG. 12 is an exploded perspective view of impulse applying means and of position control means of the device of either FIG. 3 or of FIG. 10a, the impulse applying means comprising a cam mechanism and a motor coupled thereto;

FIG. 13 is a sequence of three angular positions of the cam mechanism of FIG. 12, respectively a rest angular position, a fired angular position and an intermediate angular position between the fired angular position and the rest angular position;

FIG. 14 is a diagram of the force of a spring of a piston assembly of the cam mechanism of FIG. 12 in relation to angular positions of the cam mechanism, including the angular positions of FIG. 13;

FIG. 15 is an illustration of the interaction of a driver segment and a follower segment of the cam mechanism of FIG. 12, in the three angular positions of FIG. 13;

FIG. 16 is a schematic illustration of registering means for placing the device of either FIG. 3 or of FIG. 10a at an appropriate distance from the eye and/or for aligning the device, or an outlet thereof, with the target area on a user's eye;

FIG. 17 is a diagram exemplifying the value of a reflected beam of light as measured by a reflectance proximity sensor means of a device according to the present invention, relative to a baseline value of the sensor means, during use of the device to detect an eye blink cycle; and

FIG. 18 is a schematic representation of a subset of steps of a method for delivering a fluid formulation according to the present invention, including a step of determining that a device according to the present invention is within an appropriate distance range from an eye and a step of determining that an eye blink cycle is occurring, based on a rate of change in the intensity of the reflected beam of light of FIG. 17.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

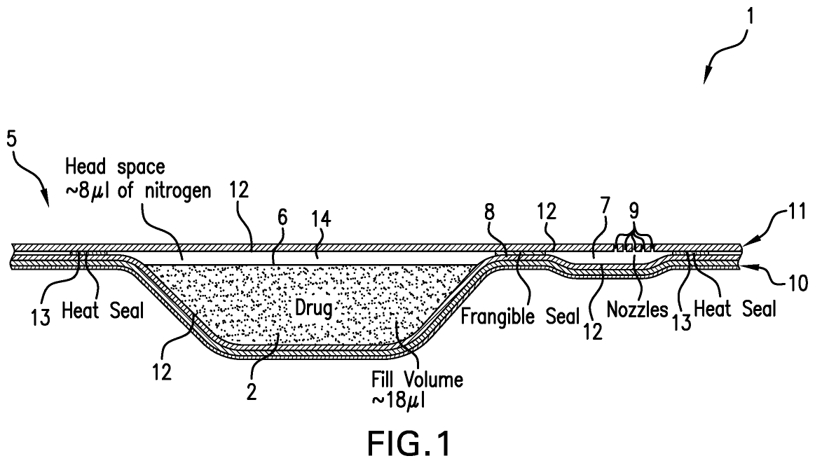

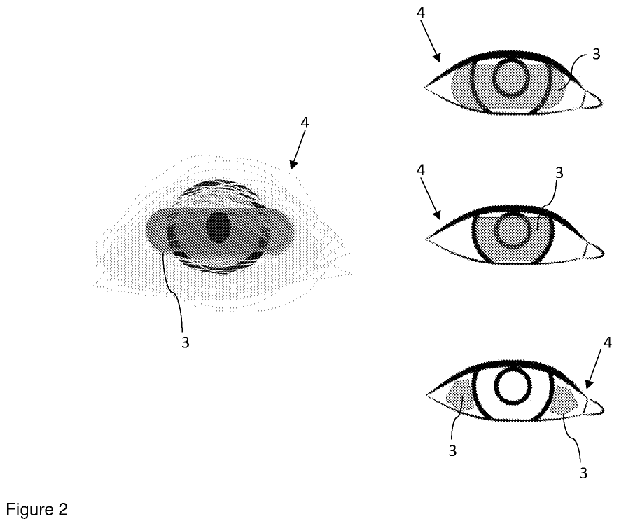

With reference to FIG. 1 and to FIG. 2, a package 1 according to the present invention contains a fluid formulation 2 to be delivered as a spray or a jet of droplets to a target area 3 on an eye 4. The package 1 contains an enclosed container 5 comprising a storage recess 6 containing the fluid formulation 2; and a delivery recess 7, adjacent to the storage recess 6. The package 1 takes substantially the form of a blister package. The storage 6 is substantially a blister cavity. A matrix of holes 9 for generating the spray and/or jet of droplets is provided on the enclosed container 5. Specifically, the holes 9 open into the delivery recess 7.

The matrix of holes 9 is configured to steer the spray and/or jet of droplets to the target area 3 on the eye 4.

The enclosed container 5 comprises a base element 10, which is cold formed to embed the storage recess 6; and a cover element 11, attached at least in part to the base element 10, closing the storage recess 6. The base element 10 and the cover element 11 further cooperate to create the delivery recess 7.

The delivery recess 7 is substantially an expansible pocket, which is created by affixing the base element 10 to the cover element 11 in a way that they are close in contact but not attached over the delivery recess area.

The enclosed container 5 comprises a permanent seal 13 between the base element 10 and the cover element 11. The permanent seal 13 is created along the outer periphery of the storage recess 6 and of the delivery recess 7.

During storage of the fluid formulation 2 in the storage recess 6, the storage recess 6 and the delivery recess 7 are separated by a fluid barrier which takes the form of a frangible seal 8. The frangible seal 8 is positioned between the base element 10 and the cover element 11.

When the package 1 is used for delivering the fluid formulation 2, the storage recess 6 is configured to expel a dose of fluid formulation 2 beyond the frangible seal 8 to the delivery recess 7. The frangible seal 8 breaks upon application of a specific pressure impulse, applied to the storage recess 6 by a device 20 according to the present invention, thus providing the desired fluid expulsion force.

As the delivery recess 7 fills with fluid formulation 2, the expansible pocket of the delivery recess 7 channels the fluid formulation 2 to the holes 9 for spray or jet delivery.

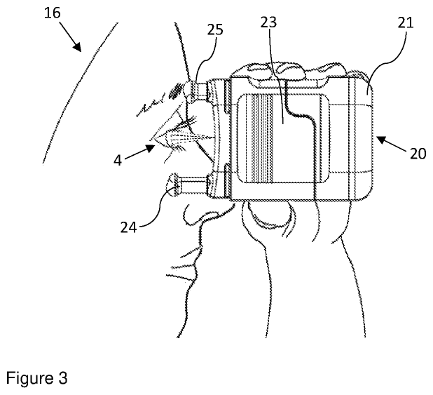

FIG. 3 shows a first embodiment of a device 20 according to the present invention, adapted to lodge the package 1, as it is employed by a user 16 for delivering a spray or a jet of droplets to a target area 3 of his eye 4.

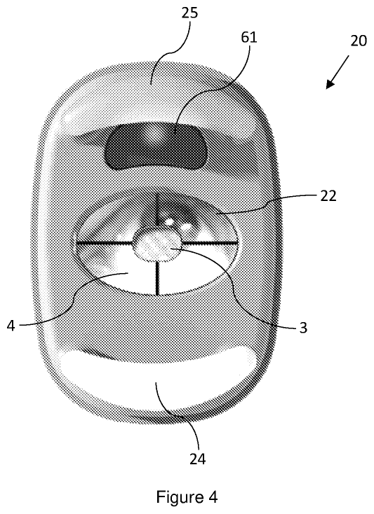

In FIG. 4, an outlet 22 of the device 20 of FIG. 3, adapted to let the spray or the jet of droplets out of the device 20, is displayed in alignment with a target area 3 on the user's eye 4.

The device 20 imparts the pressure impulse to the storage recess 6 in order to create the spray or jet of droplets out of the package 1.

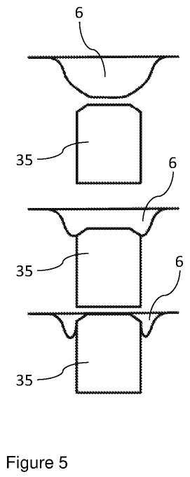

FIG. 5 shows how the storage recess 6 of the package 1, once impacted, progressively collapses by folding into itself under a pressure impulse imparted by a firing member 35 of the device 20. Thus, the force required to crush the storage recess 6 is reduced and the residual volume of fluid formulation 2 remaining in the storage recess 6 after use is minimised. Approximately, the diameter of the firing member 35 equals the diameter of the storage recess 6, less four wall thicknesses of package material.

The base element 10 and the cover element 11 are made of aluminium laminate material. The laminate material is structured in a way that the inner walls 12 of the enclosed container 5 are created by a layer of polyethylene which contacts the fluid formulation 2.

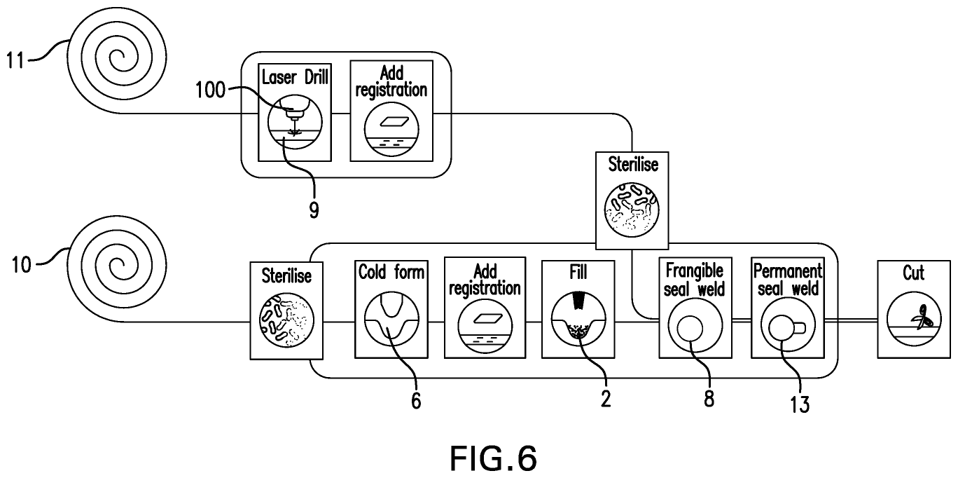

In FIG. 6, a schematic manufacturing line for the package 1 according to the present invention is shown. The package 1 is manufactured out of two rolls of cover element 11 and base element 10. The matrix of holes 9 is created on the cover element 11 by a femtosecond laser 100. As already mentioned, such a high speed laser increases throughput and allows for a low cost per blister package 1. The high speed laser also vaporises material which reduces the risk of particulates and thermal damage during drilling.

The base element 10 is first sterilised, then cold formed to create the storage recess 6. A registration is added to both the base element 10 and the cover element 11 in order to match the relative position and orientation, such that the superimposition of cover element 11 on base element 10 is executed as desired. The formed storage recess 6 is thus filled with the required volume of fluid formulation 2.

Once the cover element 11 has also been made sterile, base element 10 and cover element 11 are brought together and affixed so as to form delivery recess 7. Seals 8 and 13 are then created between the base element 10 and the cover element 11. More specifically, after the frangible seal 8 is first created around the storage recess 6, the permanent seal 13 is created along the outer periphery of the storage recess 6 and of the delivery recess 7. The permanent seal 13 and the frangible seal 8 are heat seals, created ultrasonically.

In case of a fluid formulation 2 that is oxygen sensitive, the storage recess 5 can further comprise a volume 14 (FIG. 1) of five to ten microliters of an inert gas, such as nitrogen.

The package 1 manufactured as above described is subsequently cut.

As mentioned, the unified system, or platform, for delivery of several topical ocular drug formulations according to the present invention comprises a device 20 configured to prompt the spray or jet of fluid formulation droplets out of the package 1 therein lodged.

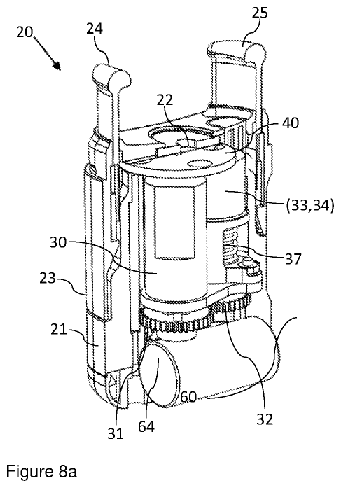

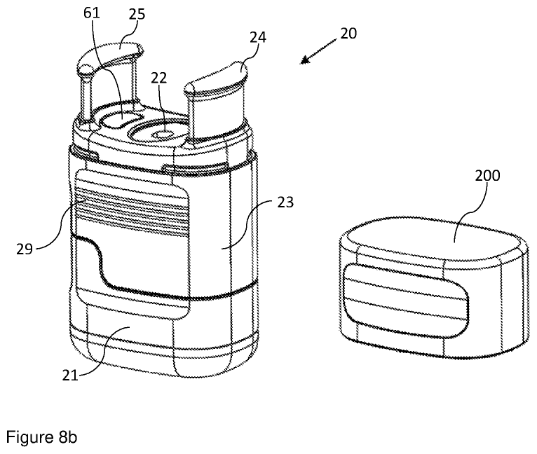

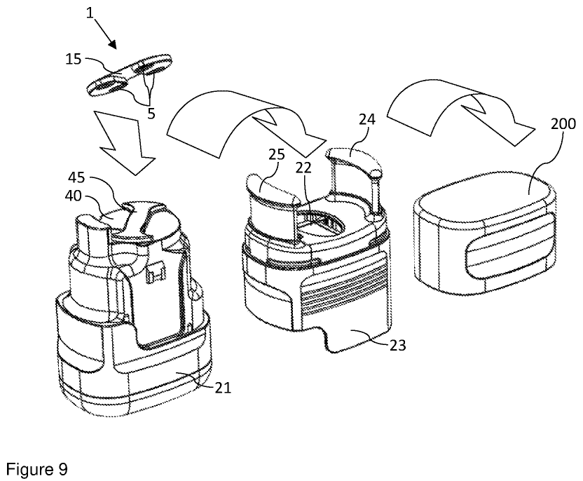

The device 20 exemplified in FIG. 3 is further portrayed in FIG. 8a, FIG. 8b and FIG. 9. Relative to such first embodiment, the device 20 is adapted to lodge a package 1 comprising a plurality of enclosed containers 5, namely four enclosed containers 5, for a daily dosing regimen consisting of two daily doses for each eye.

The device 20 comprises an external case, or shell, 21 and an outlet 22.

Internal to the case 21, a position control means comprises indexing means such as a turntable 40. Turntable 40 is provided for lodging the package 1 and for feeding in succession each of the enclosed containers 5 to a dosage station wherein a dose of fluid formulation 2 is expelled out of the enclosed container 5 to create a spray or a jet of droplets.

As portrayed in FIG. 9, the package 1 is substantially shaped as an oblong profiled disk 15 comprising the plurality of enclosed containers 5 on two extremity lobes. The special design of the package 1 allows also for improved manipulation by a user 16. The enclosed containers 5 are packed to each other on a common support of the disk and are arranged on the disk 15 so that each respective matrix of holes 9 is oriented substantially towards the center of the disk 15. The holes 9 are therefore as close as possible to the central axis of the device 20, or to the center of the outlet 22.

The impulse applying means of device 20, apt to apply a pressure impulse to a storage recess 6 of an enclosed container 5 positioned at a dosage station, comprises a cam mechanism and a motor 30 coupled thereto. The cam mechanism comprises a piston assembly comprising a driver segment 33 and a follower segment 34 resiliently biased by way of a contrast spring 37 packed therebetween.

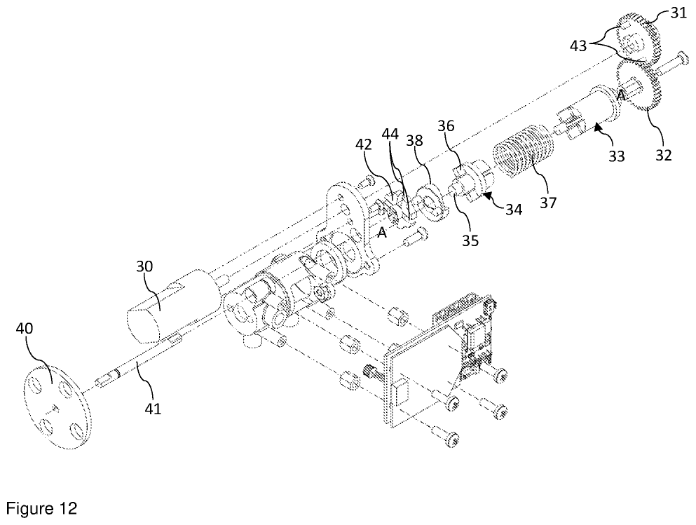

The motor 30, mounted on a motor chassis, drives a motor spur gear 31 which in turn engages a drive spur gear 32. Drive spur gear 32 rotatively drives the cam mechanism. The exact functioning of the cam mechanism and of the turntable 40 will be described in the following, with reference to FIG. 12.

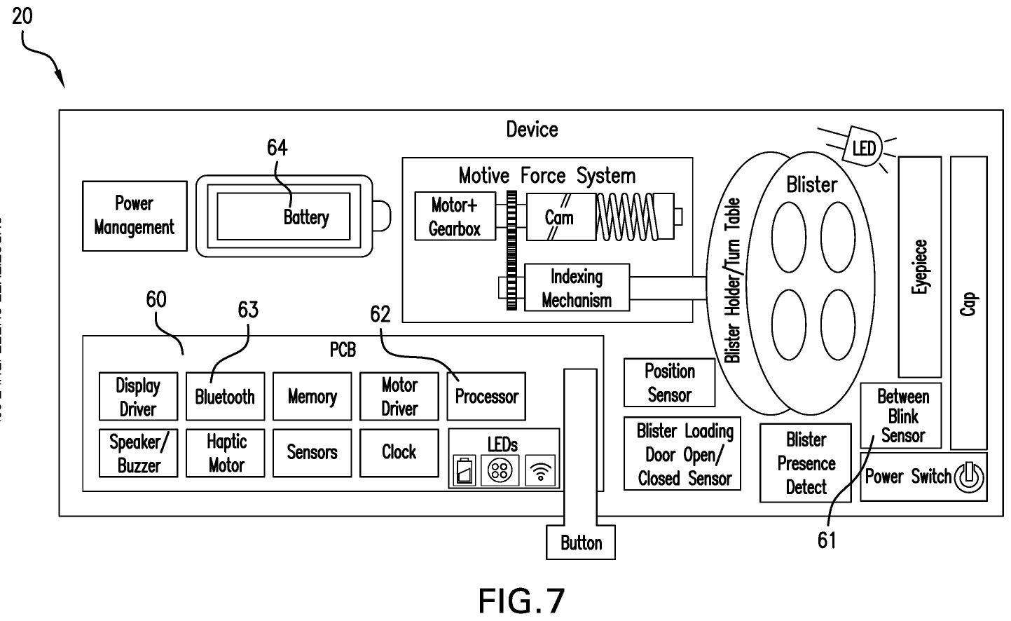

A printed circuit board 60 mechanically supports and electrically connects electronic components of an electronic circuitry of the device 20. With reference to the scheme of FIG. 7, the electronic circuitry can comprise a memory storage means, a controller, or processor, 62 for processing electric signals, sensors to deliver detection electric signals to the processor 62, wireless communication circuitry such as a Bluetooth modem. Communication circuitry can be used for communication with a mobile hand-held communication device provided with an application program executable thereon. The electronic circuitry can further comprise a display for visual information, a speaker as acoustic output element, a clock, a haptic motor for providing the device user 16 with feedback haptic sensations data transmission and/or reception.

A reflectance proximity sensor means 61, coupled to the controller 62, is configured to determine an eye blink cycle of the user 16, in order to allow effective delivery of the fluid formulation 2 during an eye opening phase of the eye blink cycle.

Battery 64 powers the electronic circuitry of the device 20. The driving function of the motor 30, as well as other functions incorporated in the device 20, is supported by the battery 64. Battery 64 can be rechargeable and its lifespan can be managed by a power management module to be maximized.

Device 20 comprises registering means for placing the device 20 at an appropriate distance from the eye 4 and/or for aligning the device 20, or its outlet 22, with the target area 3 on a user's eye 4. Relative to the embodiment of FIGS. 3, 8a, 8b and 9, the registering means comprises a removable telescopic eyecup means 23. Eyecup 23 is configured to stably rest on anatomical features surrounding the eye 4 of a user 16. To this purpose, eyecup 23 comprises two independently extractable/retractable telescopic extensions 24, 25. The eyecup 23 can be secured to the case 21, or removed therefrom, by a snap-fit engagement mechanism or similar. In order to ease a manual removal from the case 21 and a fastening thereto, the eyecup 23 can comprise grip means 29, such as spaced apart grooves providing ridges therebetween.

The outlet 22, as well as the telescopic extensions 24, 25 and the reflectance proximity sensor means 61 can be protected by way of a removable cap 200.

The turntable 40 is further provided with a profiled indexing bar 45 which functions as an indexing aid for positioning the package 1 in the delivery device 20 relative to the impulse applying means. In particular, the disk 15 and the indexing bar 45 have mutually complementary profile shapes so that they come in form-locking engagement when the disk 15 is fitted to the indexing bar 45 on the turntable 40. For loading the package 1 on the turntable 40, the cap 200 and the eyecup 23 are removed.

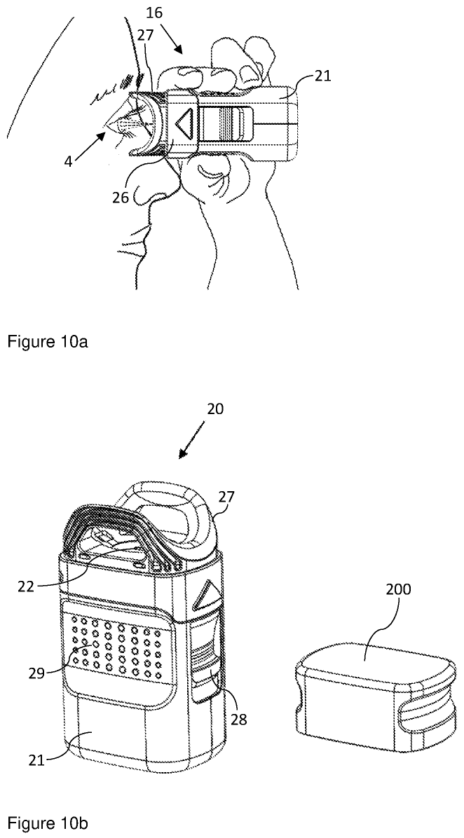

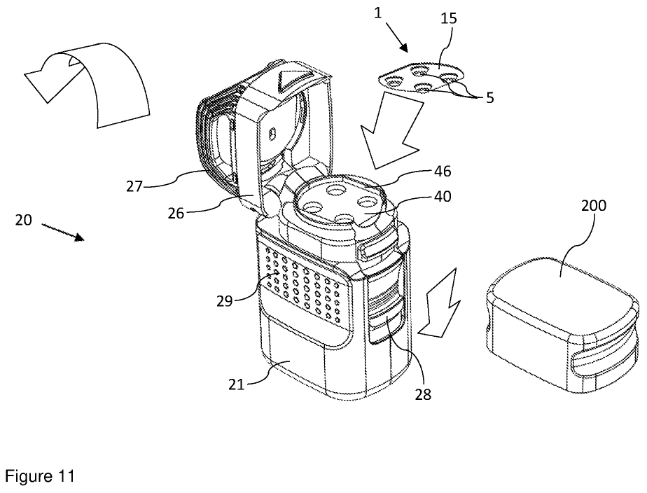

FIGS. 10a, 10b and 11 are relative to a second embodiment of the device 20 according to the present invention. Whereas the impulse applying means is substantially the same as for the first embodiment above described, the indexing means and the registering means are in part differently conceived. As for the general structure, same numbers as in the first embodiment of device 20 apply to same components.

As shown in FIG. 11, the turntable 40 is set in a profiled well 46 matching the shape of the package 1 and creating an oriented accommodation space. The package 1 is, analogously to the former embodiment, a profiled disk 15 comprising a plurality of enclosed containers 5. The package 1 is thus fittable into the profiled well 46 in two possible positions which are 180° apart.

The package 1 can be loaded thanks to a hinged eyecup 26, which allows access to the turntable 40 and is openable by way of a latch 28.

Relative to the embodiment of FIGS. 10a, 10b and 11, the registering means comprises a hinged eyecup means 26 provided with a compliant contact portion 27 configured to conform to anatomical features surrounding the eye 4 of a user 16.

For better handling by a user 16, the case 21 can comprise grip means 29, such as spaced apart rounded corrugations.

FIG. 12 clarifies on the structure of the cam mechanism and on the position control means provided in both of the above described embodiments of device 20 according to the present invention.

The cam mechanism comprises a substantially circular cam portion 38 having a circumferential profile 39 and a piston assembly. The piston assembly is resiliently biased against the profiled cam portion 38 and rotatively coupled to the profiled cam portion 38 by the motor 30.

The piston assembly comprises a driver segment 33 and a follower segment 34 which are mutually slidably engaged, to move relative to each other along a longitudinal axis A-A of the piston assembly. The driver segment 33 and the follower segment 34 are also resiliently biased by way of a contrast spring 37 packed therebetween, which makes the follower segment 34 abut against the cam portion 38.

The follower segment 34 comprises a follower member 36 and the firing member 35. The follower member 36 abuts against and follows the profile 39 of the cam portion 38. The profile 39 comprises a firing cliff.

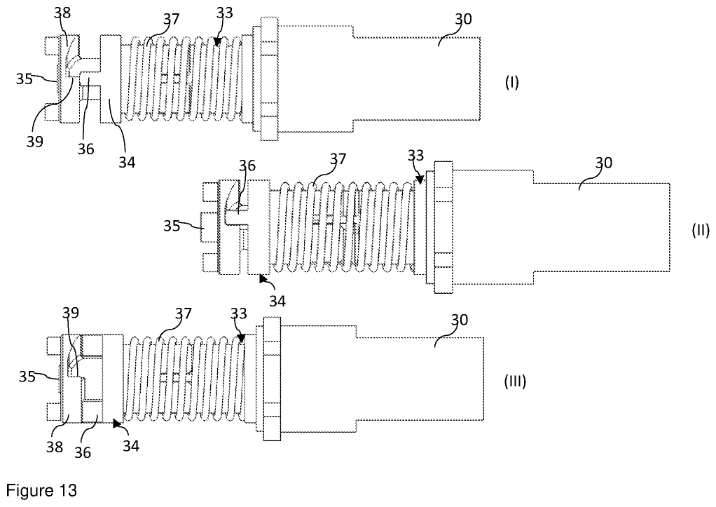

Driven in rotation by the motor 30, through engaging gear 31 and gear 32 carrying the driver segment 33, the follower segment 34 rotatively contacts, by the follower member 36, the cam portion 38 on its profile 39. Thus, with reference to FIGS. 13, 14 and 15, the impulse applying means sequentially pass from a reset angular position I to a fired angular position II.

More specifically, in the fired angular position II of the piston assembly relative to the profiled cam portion 38, the spring 37 is fully extended and the follower member 36 goes off the firing cliff of the profile 39. Consequently, the firing member 35 of the piston assembly axially advances and applies a pressure impulse on an enclosed container 5 of the package 1 positioned at a dosage station. When the follower member 36 goes off the firing cliff of the profile 39, the movement and the consequent noise are dampened by the progressive crushing of the enclosed container 5. This ensures that the user 16 does not experience unpleasant noises or abrupt tactile sensations.

In the reset angular position I of the piston assembly relative to the profiled cam portion 38, the spring 37 is compressed and the follower member 36 comes to rest on the profile 39 keeping above the firing cliff by a given angular distance. Consequently, the firing member 35 of the piston assembly is completely retracted and automatically brought to a reset position for a subsequent firing.

Substantially between angular positions III of the piston assembly relative to the profiled cam portion 38 wherein, after firing, the spring 37 has been newly fully compressed and the firing member 35 is retracted, up to the reset angular position I, the turntable 40 comes to be rotatively coupled to the motor 30 of the impulse applying means and is thereby driven in rotation to bring a next enclosed container 5 to the dosage station.

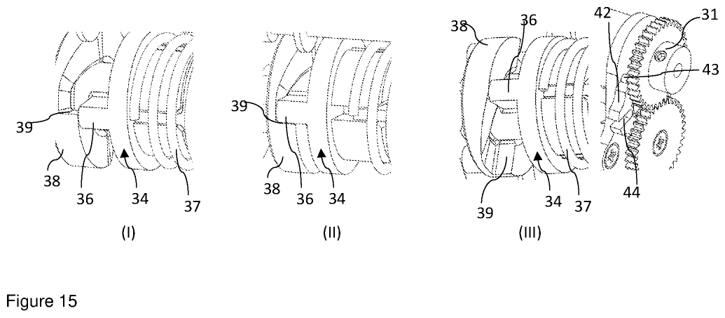

In fact, with special reference to FIGS. 12 and 15, over angular positions III where the piston assembly comes to be fully retracted and the spring is fully compressed, a Geneva wheel 42 axially approaches motor spur gear 31 to an extent that pins 43 of the motor spur gear 31 come in engagement with slots 44 of the Geneva wheel 42. Thus, the Geneva wheel 42 communicates the rotation of the motor 30 to the turntable 40 by way of an indexing shaft 41.

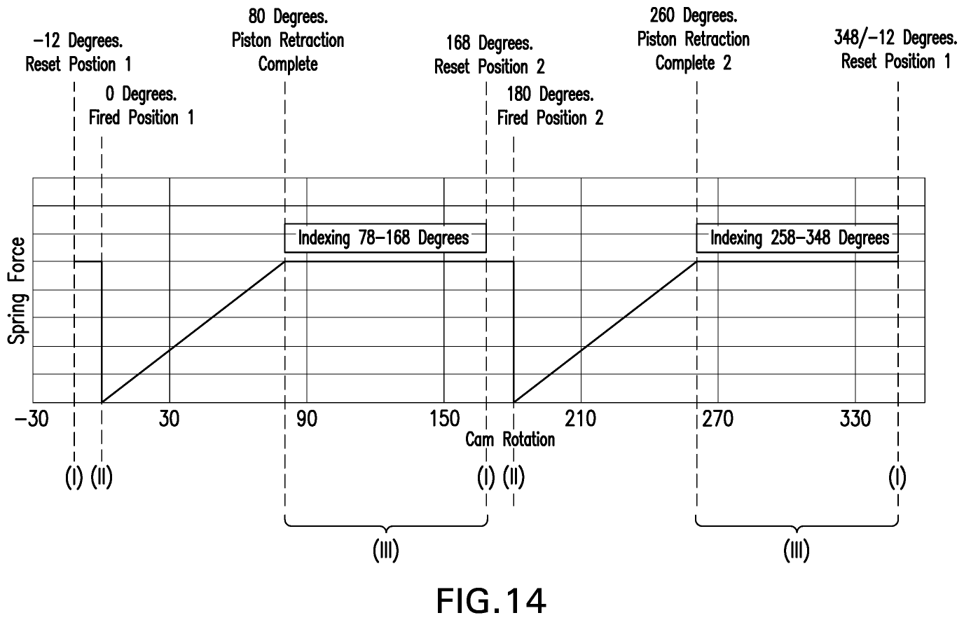

With reference to FIG. 14, for instance, the follower member 36, when in the configuration corresponding to the first reset angular position I, remains about 12° before the first fired angular position II. If the first fired angular position II is assumed at 0°, then the first reset angular position I can correspond to −12°. Advantageously, such a small angular offset allows for a prompt activation of the impulse applying means once the delivery of the fluid formulation is needed, while ensuring that no accidental delivery happens as a result of external shocks to the device 20.

After the firing, progressively the piston assembly is retracted and the spring 37 is compressed, up to about 80° when the piston assembly is fully retracted and the spring 37 is fully compressed. Just about when the spring 37 comes to exert its maximum compression force, the turntable 40 is put in rotation and indexing is activated, substantially between 78° and up to the next, second reset angular position I happening around 168°. The piston assembly is then ready for a successive, second firing, at an angular position II corresponding to 180°.

FIG. 14 exemplifies almost a full rotation of the cam mechanism by 360°.

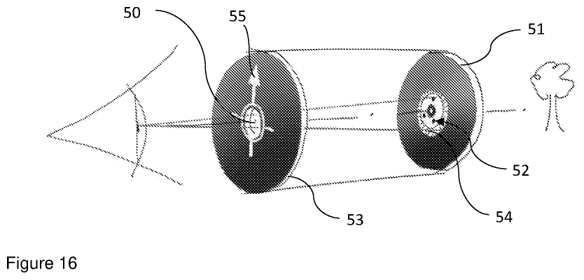

FIG. 16 shows an example of registering means comprising an offset viewing orifice 50 substantially aligned with the central axis of the outlet 22 of the device 20; and a backplate 51 comprising visual cues 52. The offset viewing orifice 50 and the backplate 51 are configured so that at an appropriate distance and/or in an aligned state the visual cues 52 are visible through the offset viewing orifice 50 in a predefined pattern and a predefined relation thereto. In particular, on the backplate 51 are three dots 52 surrounded by a circle of a different colour 54. The user 16 is instructed to ensure that all three dots 52 are visible just on the edge of the viewing orifice 50, to ensure that the device 20 is positioned on the correct axis, that is the device 20, or its outlet 22, is aligned with the target area 3 on his eye 4. This could be also be achieved differently, e.g. with two circular shapes or two triangular shapes. If the differently coloured circle 54 encircling dots 52 is visible through the viewing orifice 50, this is an indication to the user 16 that the device 20 has been brought too close to his eye 4.

The offset viewing orifice 50 is integrated in a front plate 53 which comprises further visual cues for aligning the device 20 horizontally, such as an arrow 55.

As mentioned, in order to allow effective delivery of the fluid formulation 2 during an eye opening phase of the eye blink cycle, a reflectance proximity sensor means 61, coupled to the controller 62, is employed to determine an eye blink cycle of the user 16.

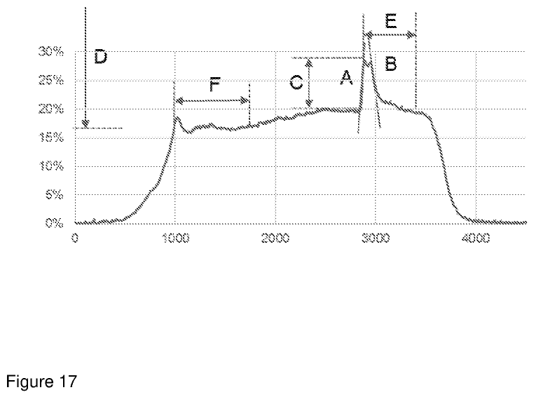

In FIG. 17, a diagram exemplifies the value of a reflected beam of infrared light as measured by a reflectance proximity sensor means 61 of the device 20, relative to a baseline value of the sensor means, during use of the device 20 to detect an eye blink cycle. Threshold values A, B, C, D, E and F are explained below.

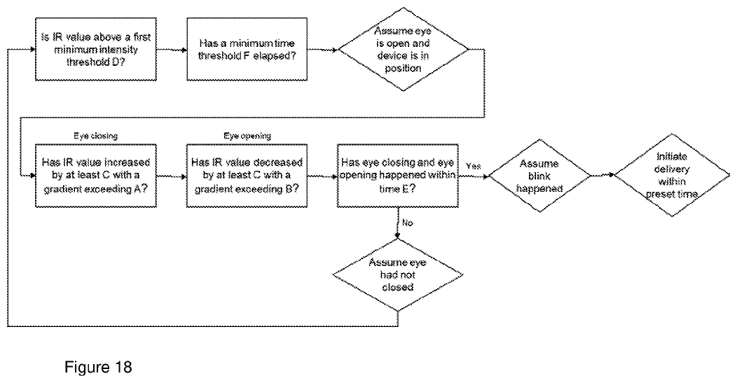

FIG. 18 is a schematic representation of a subset of steps of a method for delivering a fluid formulation according to the present invention, as implemented by a corresponding algorithm. Shown are a step of determining that a device 20 is within an appropriate distance range from an eye and a step of determining that an eye blink cycle is occurring, based on a rate of change in the intensity of the reflected beam of light as shown in FIG. 17. The method is based on threshold values A, B, C, D, E and F as shown in FIG. 17.

The method for delivering a fluid formulation 2 according to the present invention comprises steps of sending out a beam of light to the user's eye 4 by an emitter unit of the reflectance proximity sensor means 61 and detecting a corresponding beam of light reflected therefrom by a receiver unit of the reflectance proximity sensor means 61.

Thereafter, it is determined if the device 20 is in position, that is within an appropriate distance range from the eye 4 and/or aligned with the target area 3 on the user's eye 4, or with a pupil thereof. For this purpose, as shown in FIG. 17, the method comprises a step of measuring a value of the reflected beam of light. If the value of the reflected beam of light is lower than a first minimum intensity threshold value D, the algorithm assumes the device is not within an appropriate distance range from the eye and does not proceed to the next step of determining a rate of change in the reflected beam of light. If, instead, the value of the reflected beam of light is higher than the first minimum intensity threshold value D, it is determined if the value of the reflected beam of light maintains above the first minimum intensity threshold value D for a time longer than a minimum time threshold F. If the value of the reflected beam of light does not maintain above the first minimum intensity threshold value D for a time longer than the minimum time threshold F, the algorithm does not proceed to the next step of determining a rate of change in the reflected beam of light. If, on the contrary, the value of the reflected beam of light maintains above the first minimum intensity threshold value D for a time longer than the minimum time threshold F, then the algorithm assumes that the device 20 is in position, with the user's eye 4 open.

Under these conditions, the algorithm then determines that an eye blink cycle is occurring, based on a rate of change in the intensity of the reflected beam of light. Determining that an eye blink cycle is occurring comprises the step of determining an eye closing phase, followed by a step of determining an eye opening phase.

Determining the eye closing phase comprises the steps of measuring the current the value of the reflected beam of light; measuring the minimum value of the reflected beam of light within a refresh time equal to the maximum length of time within minimum and maximum values of the reflected beam of light are updated; and calculating a difference between the current value and the minimum value of the reflected beam of light as above measured to establish that the difference is at least a threshold value C. The algorithm finally concludes that an eye closing phase is happening if it also determines that the rate of positive change is higher than a minimum positive gradient threshold A.

Determining the eye opening phase of the eye blink cycle comprises the steps of measuring the current the value of the reflected beam of light; measuring the maximum value of the reflected beam of light within a refresh time equal to the maximum length of time within which minimum and maximum values of the reflected beam of light are updated; and calculating a difference between the current value and the maximum value of the reflected beam of light as above measured to establish that the difference is at least a threshold value C.

The algorithm finally concludes that an eye opening phase is happening if it also determines that the rate of negative change is higher than a minimum negative gradient threshold B; combined with verifying that the conditions above are met within a preset maximum blink time E from the eye closing phase. If this is the case, then the algorithm assumes that the blink cycle is actually consequently happening and that the eye is currently open as a result of an eye opening phase. During the eye opening phase, a delivery activation signal is transmitted to the impulse applying means and the delivery is instructed of the fluid formulation 2 as a spray or a jet of droplets to the target area 3 of the eye 4 within a predefined time from the end of the eye opening phase.

If an eye opening phase cannot be confirmed, then the algorithm assumes the eye had not originally closed and reassess if the device 20 is in position as above described.

This description and the accompanying drawings that illustrate aspects and embodiments of the present invention should not be taken as limiting the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the FIGS. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. Method for delivering a fluid formulation (2) as a spray or a jet of droplets to a target area (3) on an eye (4) of a user (16) by a device (20),
wherein the device (20) comprises extraction means (30, 31, 32, 33, 34, 35, 36, 37, 38, 39) for extracting the fluid formulation (2) from an enclosed container (5) lodged in, or coupled to, the device (20);
comprising the steps of:
sending out a beam of light to the user's eye (4);
detecting a corresponding beam of light reflected therefrom;
determining that the device (20) is within an appropriate distance range from the eye (4) and/or aligned with the target area (3) on the user's eye (4), or with a pupil thereof;
determining that an eye blink cycle is occurring, based on a rate of change in the intensity of the reflected beam of light;
determining an eye opening phase of the eye blink cycle;
during the eye opening phase, transmitting a delivery activation signal to the extraction means (30, 31, 32, 33, 34, 35, 36, 37, 38, 39);
delivering the fluid formulation (2) as a spray or a jet of droplets to the target area (3) of the eye (4) within a predefined time from the end of the eye opening phase.

2. The method of claim 1, wherein determining that the device (20) is within an appropriate distance range from the eye (4) comprises the steps of:
measuring a value of the reflected beam of light;
if the value of the reflected beam of light is lower than a first minimum intensity threshold value (D), assuming the device is not within an appropriate distance range from the eye and not proceeding to the next step of determining a rate of change in the reflected beam of light;
if the value of the reflected beam of light is higher than the first minimum intensity threshold value (D), determining if the value of the reflected beam of light maintains above the first minimum intensity threshold value (D) for a time longer than a minimum time threshold (F);
if the value of the reflected beam of light does not maintain above the first minimum intensity threshold value (D) for a time longer than the minimum time threshold (F), not proceeding to the next step of determining a rate of change in the reflected beam of light;
if the value of the reflected beam of light maintains above the first minimum intensity threshold value (D) for a time longer than the minimum time threshold (F), then determining a rate of change in the reflected beam of light.

3. The method of claim 1, wherein determining that an eye blink cycle is occurring comprises the step of determining an eye closing phase, followed by a step of determining an eye opening phase,
wherein determining the eye closing phase comprises the steps of:
measuring the current value of the reflected beam of light;
measuring the minimum value of the reflected beam of light within a refresh time equal to the maximum length of time within minimum and maximum values of the reflected beam of light are updated;
calculating a difference between the current value and the minimum value of the reflected beam of light as above measured and establishing that the difference is at least a threshold value (C);
determining that the rate of positive change is higher than a minimum positive gradient threshold (A).

4. The method of claim 3, wherein determining the eye opening phase of the eye blink cycle comprises the steps of:
measuring the current the value of the reflected beam of light;
measuring the maximum value of the reflected beam of light within a refresh time equal to the maximum length of time within which minimum and maximum values of the reflected beam of light are updated;
calculating a difference between the current value and the maximum value of the reflected beam of light as above measured and establishing that the difference is at least a threshold value (C);
determining that the rate of negative change is higher than a minimum negative gradient threshold (B);
verifying that the conditions above are met within a preset maximum blink time (E) from the eye closing phase.
